(19) United States
(12) Patent Application Publication
(10) Pub. No.: US 2002/0151876 A1
(43) Pub. Date: Oct. 17, 2002

(54) Devices and methods for management of bone density

(76) Inventor: Tai-Wah Chan, Palo Alto, CA (US)

Correspondence Address: DURECT CORPORATION, 10240 BUBB ROAD, CUPERTINO, CA, 95014, US

(21) Appl. No.: 10/071,821

(22) Filed: Feb. 7, 2002

Related U.S. Application Data
(60) Provisional application No. 60/267,323, filed on Feb. 7, 2001.

(51) Int. Cl. A61K 9/22; A61F 2/00

(52) U.S. Cl. 604/891.1; 424/423

(57) ABSTRACT

The invention features devices and methods for the delivery of a formulation to an individual to stabilize or increase bone mass by increasing bone deposition and/or decreasing bone resorption. In the present invention, a drug formulation comprising a bisphosphonate is provided parenterally in a sustained release dosage form, e.g., as an injected matrix or stored within a drug delivery device. In a specific embodiment, the dosage form may be implanted or injected into a site in the body (i.e., implantation site) and a conduit, e.g. a catheter, can be used to transport the formulation from the dosage form for release at a site in the body distal from the implantation site.

This application claims priority to U.S. Provisional Application Serial No. 60/267,323 filed Feb. 7, 2001.

### FIELD OF THE INVENTION

The invention relates to devices and methods for the management of bone mass levels, e.g., increasing bone mass and/or preventing bone loss in a subject.

### BACKGROUND OF THE INVENTION

Osteoporosis

Osteoporosis is a disease characterized by low bone mass and structural deterioration of bone tissue, leading to bone fragility and an increased susceptibility to fractures of the hip, spine, and wrist. Osteoporosis is a major public health threat for 28 million Americans, 80% of whom are women. In the U.S. today, 10 million individuals already have osteoporosis and 18 million more have low bone mass, placing them at increased risk for this disease. Currently, one out of every two women and one in eight men over the age of 50 will have an osteoporosis-related fracture in their lifetime. Osteoporosis is responsible for more than 1.5 million fractures annually, including 300,000 hip fractures, and approximately 700,000 vertebral fractures, 250,000 wrist fractures, and more than 300,000 fractures at other sites. Estimated national direct expenditures (hospitals and nursing homes) from osteoporosis and related fractures is $14 billion each year.

There are two categories of osteoporosis: primary osteoporosis and secondary osteoporosis. Primary osteoporosis can be further divided into three types. Type 1, or postmenopausal osteoporosis, characterized by the disproportionate loss of trabecular bone, is associated with fractures at sites rich in cancellous bone such as the vertebral body and distal radius. Type 2, or age-associated osteoporosis, affects all skeletal sites with both cortical and cancellous bone such as the proximal femur, and is a result of senile decline in bone mass. Type 3 is idiopathic osteoporosis which affects premenopausal women as well as middle-aged and young men. Secondary osteoporosis, the second category of osteoporosis, can be caused by an identifiable agent such as glucocorticoids, or by a disease such as hyperthyroidism or myeloma. Although there are many causes of osteoporosis, the most common cause is estrogen deficiency in postmenopausal women. See, e.g., Riggs B L and Melton L J III, N Engl J Med; 314: 1676-84(1986); Melton L J III and Riggs B L. Clinical spectrum. In: Osteoporosis: etiology, diagnosis and management. New York: Raven Press, 1988:155-79. Osteoporotic fracture, which is the major health consequence of this condition, may occur at any skeletal site. However, the primary sites are the spine, hip (proximal femur) and distal forearm.

Bisphosphonates

Bisphosphonates have emerged as a novel class of non-hormonal compounds available to treat osteoporosis. Bisphosphonates are stable analogues of pyrophosphate which bind to the bone surface and inhibit osteoclastic activity. The predominant means by which a bisphosphonate reduces bone turnover both in vitro and in vivo appears to be through the local, direct antiresorptive. Bilke D D et al., J. Bone and Mineral Res., 9:1777-1787. Bisphosphonates suppress the migration of osteoclast precursors onto the bone and their subsequent transformation into the mature resorbing osteoclasts. Bisphosphonates also have the ability to increase osteoblast proliferation and cytodifferentiation in a dose-dependent manner in cultures treated with bisphosphonates. Reinholz G G, Cancer Res 60:6001-7 (2000).

As a group, the bisphosphonates offer several advantages over estrogens in treating osteoporosis. They are bone-tissue specific, have minimal side effects, cause no known risk of carcinogenesis, have antiresorptive efficacy that is equivalent to or greater than estrogens and there is prospective evidence for a reduction in incident vertebral fractures.

However, bisphosphonates may have other effects, such that use of bisphosphonate therapy presents considerable risk of deleterious side-effects to patients. Side effects from bisphosphonates (e.g., alendronate) include abdominal or musculoskeletal pain, nausea, heartburn, or irritation of the esophagus. Compliance with bisphosphonate therapy is often a problem due certain problems associated with administration. For example, orally administered bisphosphonates should be taken on an empty stomach, and a subject must wait in an upright position for at least one-half hour, or preferably one hour, before ingesting any other food, beverage, or medication. Bisphosphonates may also trigger the development of mucosal injury and possible ulceration of the upper gastrointestinal tract. Lichtenberger L M et al., Dig Dis Sci. 45:1792-801 (2000). In addition, certain bisphosphonates can cause severe local reactions and thrombophlebitis upon administration as a bolus injection.

Drug Delivery Devices

Well-known drug delivery devices include osmotic, mechanical or electromechanical infusion pumps such as those described in, for example, U.S. Pat. Nos. 4,692,147; 4,360,019; 4,487,603; 4,360,019; 4,725,852, and the like. Osmotically-driven pumps (such as the DUROST™ osmotic pump) are described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; 5,985,305; 5,728,396 and WO 97/27840.

Another well-known drug delivery device is the “depot” which is an injectable biodegradable sustained release device that is generally non-containerized and that may act as a reservoir for a drug, and from which a drug is released. Depots include polymeric and non-polymeric materials, and may be solid, liquid or semi-solid in form. For example, a depot as used in the present invention may be a high viscosity liquid, such as a non-polymeric non-water-soluble liquid carrier material, e.g., Sucrose Acetate Isobutyrate (SAIB) or another compound described in U.S. Pat. Nos. 5,747,058 and 5,968,542, both expressly incorporated by reference herein. For reference, please refer generally to “Encyclopedia of Controlled Drug Delivery” 1999, published by John Wiley & Sons Inc, edited by Edith Mathiowitz.

There has been extensive research in the area of biodegradable controlled release systems for bioactive compounds. Biodegradable matrices for drug delivery are useful because they obviate the need to remove the drug-depleted device. The most common matrix materials for drug delivery are polymers. The field of biodegradable polymers has developed rapidly since the synthesis and biodegradability of polylactic acid was reported by Kulkarni et al., in 1966 (“Polylactic acid for surgical implants,” Arch. Surg., 93:839). Examples of other polymers which have been reported as useful as a matrix material for delivery devices include polyanhydrides, polyesters such as polyglycolides and polylactide-co-glycolides, polyamino acids such as polylysine, polymers and copolymers of polyethylene oxide, acrylic terminated polyethylene oxide, polyamides, polyurethanes, polyorthoesters, polyacrylonitriles, and polyphosphazenes. See, for example, U.S. Pat. Nos. 4,891,225 and 4,906,474 to Langer (polyanhydrides), U.S. Pat. No. 4,767,628 to Hutchinson (polylactide, polylactide-co-glycolide acid), and U.S. Pat. No. 4,530,840 to Tice, et al. (polylactide, polyglycolide, and copolymers).

Degradable materials of biological origin are well known, for example, crosslinked gelatin. Hyaluronic acid has been crosslinked and used as a degradable swelling polymer for biomedical applications (U.S. Pat. No. 4,957,744 to Della Valle et al.; (1991) “Surface modification of polymeric biomaterials for reduced thrombogenicity,” Polym. Mater. Sci. Eng., 62:731-735).

Biodegradable hydrogels have also been developed for use in controlled drug delivery as carriers of biologically active materials such as hormones, enzymes, antibiotics, antineoplastic agents, and cell suspensions. Temporary preservation of functional properties of a carried species, as well as the controlled release of the species into local tissues or systemic circulation, have been achieved. See for example, U.S. Pat. No. 5,149,543 to Cohen. Proper choice of hydrogel macromers can produce membranes with a range of permeability, pore sizes and degradation rates suitable for a variety of applications in surgery, medical diagnosis and treatment.

Many dispersion systems are currently in use as, or being explored for use as, carriers of substances, particularly biologically active compounds. Dispersion systems used for pharmaceutical and cosmetic formulations can be categorized as either suspensions or emulsions. Suspensions are defined as solid particles ranging in size from a few nanometers up to hundreds of microns, dispersed in a liquid medium using suspending agents. Solid particles include microspheres, microcapsules, and nanospheres. Emulsions are defined as dispersions of one liquid in another, stabilized by an interfacial film of emulsifiers such as surfactants and lipids. Emulsion formulations include water in oil and oil in water emulsions, multiple emulsions, microemulsions, microdroplets, and liposomes. Microdroplets are unilamellar phospholipid vesicles that consist of a spherical lipid layer with an oil phase inside, as defined in U.S. Pat. Nos. 4,622,219 and 4,725,442 issued to Haynes. Liposomes are phospholipid vesicles prepared by mixing water-insoluble polar lipids with an aqueous solution. The unfavorable entropy caused by mixing the insoluble lipid in the water produces a highly ordered assembly of concentric closed membranes of phospholipid with entrapped aqueous solution.

U.S. Pat. No. 4,938,763 to Dunn, et al., discloses a method for forming an implant in situ by dissolving a non-reactive, water insoluble thermoplastic polymer in a biocompatible, water soluble solvent to form a liquid, placing the liquid within the body, and allowing the solvent to dissipate to produce a solid implant. The polymer solution can be placed in the body via syringe. The implant can assume the shape of its surrounding cavity. In an alternative embodiment, the implant is formed from reactive, liquid oligomeric polymers which contain no solvent and which cure in place to form solids, usually with the addition of a curing catalyst.

As is evident from the above, there is a great need for devices and methods for effective and practical long-term management of bone density. The present invention addresses this problem.

### SUMMARY OF THE INVENTION

The invention features devices and methods for the delivery of a formulation to an individual to stabilize or increase bone mass by increasing bone deposition and/or decreasing bone resorption. In the present invention, a drug formulation comprising a bisphosphonate is provided parenterally in a sustained release dosage form, e.g., as a SAIB “depot” injectable or stored within a drug delivery device. Once released from the dosage form, the drug formulation enters the systemic circulation and is transported to the site of action in the body to modulate bone deposition. Alternatively, in another embodiment, the dosage form may be implanted or injected into a site in the body proximal to the desired site of action and the drug formulation may reach the site by diffusion. In yet another embodiment, the dosage form may be implanted or injected into a site in the body (i.e., implantation site) and a conduit, e.g. a catheter, can be used to transport the formulation from the dosage form for release at a site in the body distal from the implantation site, (e.g., the spine, hip, etc).

In one aspect, the invention features methods of maintaining or increasing bone density by delivery of a formulation comprising a bisphosphonate to the subject from a sustained release dosage form. The formulation can be introduced to a subject via any parenteral delivery system with the ability to provide release of the formulation for a pre-selected period of time. Exemplary delivery methods include, but are not limited to, injectable sustained release dosage forms such as a depo-type preparation or an injectable formulation containing a sustained-release particulate preparation, e.g., a formulation having a structure of microspheres or microcapsules.

In an exemplary embodiment, delivery of the formulation is substantially continuous, and can be for a pre-selected administration period that can range from several days to years, or from about 4 weeks to 12 months.

In a particular aspect, the invention features devices for and methods of treating osteoporosis or other disorders associated with an increased risk of bone fracture comprising the steps of implanting a drug delivery device at an implantation site in the body of a subject, where the drug delivery device is capable of sustained drug release. A formulation comprising a bisphosphonate can be introduced from the device to a delivery site in an amount effective to reduce risk of fracture and/or allow an increase in or maintenance of bone deposition. A long-term release formulation for use in such a device can be, e.g., contained in a reservoir solubilized or suspended in a vehicle or impregnated within a matrix within the drug delivery device.

The drug delivery device can be based on, for example, diffusive, erodible or convective systems, e.g., osmotic pumps, biodegradable implants, electrodiffusion systems, electrochemical systems, electroosmosis systems, vapor pressure pumps, electrolytic pumps, effervescent pumps, piezoelectric pumps, erosion-based systems, or electromechanical systems. A drug formulation comprising a bisphosphonate is stored within a drug delivery device (e.g., contained in a reservoir or impregnated within a matrix within the controlled drug delivery device). The drug formulation comprises an amount of drug sufficient for treatment and is stable at body temperatures (e.g., no unacceptable degradation) for the entire pre-selected treatment period. The drug delivery devices store the drug formulation safely without harming the surrounding tissue, provide sufficient protection from bodily processes to prevent unacceptable degradation of the formulation, and release the drug formulation in a controlled fashion at a therapeutically effective rate to increase bone mass, either locally (e.g., at the hip or spine) or systemically, i.e. an overall increase in bone mass. In use, the drug delivery device is implanted in the subject's body at an implantation site, and the drug formulation is released from the drug delivery device to a delivery site. The delivery site may be the same as, near, or distant from the implantation site. Once released at the delivery site, the drug formulation enters the systemic circulation and is transported to the site of action in the body to modulate bone density.

In another particular aspect, the invention features methods of treating a subject at risk for bone fracture by systemic delivery of a formulation comprising a bisphosphonate (e.g., pamidronate) to the subject via an implantable drug delivery device, where such formulation is delivered at a rate and/or concentration sufficient to modulate bone density in the subject. In specific embodiments, the formulation can be administered at a rate of from about 0.1 μg per hour to 200 μg per hour for a period of at least a week, and can be delivered for a period of at least about a month, and or at least about six months.

In another aspect, the invention features devices for and methods of conditions arising from disease such as osteoporosis and Paget's Disease comprising the steps of implanting a drug delivery device at an implantation site in the body of a subject, where the drug delivery device is capable of controlled drug release; and delivering a formulation comprising a bisphosphonate from the device to a delivery site for delivery to the systemic circulation in an amount effective to increase overall bone density in the subject.

In another aspect, the invention features local administration of a bisphosphonate at a region at risk for fracture, e.g., the hip or spine.

In various exemplary embodiments of the invention and various aspects thereof, drug of the drug formulation administered is delivered at a low dose rate due the potency of the subject drugs, e.g., from about 0.01 μg/hr or 0.1 μg/hr, 0.25 μg/hr, 1 μg/hr, 10 μg/hr, 50 μg/hr, 100 μg/hr, 250 μg/hr, 500 μg/hr, 750 μg/hr, and generally up to about 1000 μg/hr. Specific ranges of amount of drug delivered will vary depending upon, for example, the potency and other properties of the drug used and the therapeutic requirements of the subject. In one specific embodiment, the formulation is delivered at a rate of from about 100 μg/hr, 250 μg/hr, 500 μg/hr, 750 μg/hr, and generally up to about 1000 μg/hr.

In another exemplary embodiment, the drug formulation is delivered at a low volume rate e.g., a volume rate of from about 0.0001 ml/day to about 1 ml/day.

A primary object of the invention is provide a method for convenient, long-term management of bone mass loss and/or an increase in bone density in a treated individual.

One advantage of the invention is that the devices and methods described herein provide effective management of bone density by administration of a relatively small quantity of a bisphosphonate. Given the long-term, chronic effects of loss of bone mass, this advantage is of considerable benefit for relatively long term (e.g., 1-4 months) dosage regimes. Furthermore, the method may be more cost-effective than current prescription drugs, and thus may make treatment for reduced bone mass available to a broader population.

Another advantage of the present invention is that treatment according to the methods of the invention can be used to prevent fractures and/or relieve symptoms of fractures and skeletal deformities.

Another advantage of the invention is that the devices and methods described herein provide means to stabilize or increase bone mass, and thus may be used to prevent complications associated with reduced bone mass in an individual at risk for such complications, e.g., a patient suffering from or at risk for osteoporosis.

Another advantage is that the formulations of the invention are non-toxic, and do not cause unacceptable irritation upon parenteral delivery to a subject.

Another advantage of the invention is that the devices and methods described herein provide means for elderly subjects to maximize physical function and increase quality of life.

The present invention is also advantageous in that it can provide for safe, effective therapy while minimizing the risk of undesirable side effects. This can be accomplished by delivery of bisphosphonates at low dose and/or low volume rates to avoid or eliminate the overdosing and underdosing inherent with bolus administration, and also are not associated with detectable thrombophlebitis. In addition, the methods of the invention allow bisphosphonate administration without the gastrointestinal side effects associated with oral dosing.

Another advantage of the invention is that the invention can be used to deliver relatively small quantities of bisphosphonates accurately and precisely. Thus, the invention allows for the convenient use of these drugs for treatment, and particularly for the delivery of small amounts locally, e.g., to an area at particular risk of fracture.

Another notable advantage of the invention is that the implanted device increases patient compliance with a prescribed therapeutic regimen because dosing does not require patient action, for example in following a precise regimen. This is particularly important since compliance is particularly difficult to achieve in prophylactic treatment before the onset of disease or symptom and since the population that needs treatment often has difficulty with compliance, e.g., the infirmed, the elderly and/or people with gastrointestinal difficulties. Improved compliance will provide an improved therapeutic outcome in the patient.

Another advantage of the invention is that a bisphosphonate can be delivered in a patterned fashion, e.g., continuously, with such accuracy and precision and at such low quantities as to permit long-term use of such compounds to treat overall loss in bone mass.

A further advantage is that a therapeutically effective dose of a bisphosphonate can be delivered at such relatively low volume rates, e.g., from about 0.001 ml/day to 1 ml/day so as to minimize tissue disturbance or trauma near the site where the formulation is released.

Another advantage of the invention is that substantially continuous delivery of small quantities of bisphosphonate (e.g., alendronate or risedronate) is effective in long-term (e.g., chronic) administration (e.g., from several weeks or from about 1 to 12 months or more).

Yet another advantage is that the invention provides for precise delivery of the selected bisphosphonate, thus allowing delivery of lower doses and/or for delivery of precisely metered doses at consistent delivery volume rates (e.g., on the order of microliters to milliliters per hour).

These and other objects, advantages and features of the present invention will become apparent to those persons skilled in the art upon reading the details of the methodology and compositions as more fully set forth below.

### BRIEF DESCRIPTION OF THE DRAWINGS

FIG. 1 illustrates systemic delivery of the drug formulation using an implanted drug delivery device.

FIG. 2 is a perspective view of an exemplary drug delivery device useful in the present invention.

FIG. 3 is a cut-away view of an exemplary drug delivery device comprising a catheter.

### DESCRIPTION OF THE PREFERRED EMBODIMENTS

Before the present device and methods for bone density maintenance are described, it is to be understood that this invention is not limited to the specific methodology, devices, therapeutic formulations, and syndromes described as such may, of course, vary. It is also to be understood that the terminology used herein is for the purpose of describing particular embodiments only, and is not intended to limit the scope of the present invention that will be limited only by the appended claims.

It must be noted that as used herein and in the appended claims, the singular forms “a”, “and”, and “the” include plural referents unless the context clearly dictates otherwise. Thus, for example, reference to “a drug delivery device” includes a plurality of such devices and reference to “the method of delivery” includes reference to equivalent steps and methods known to those skilled in the art, and so forth.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the compositions and methodologies which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

Definitions

The terms “increased bone mass”, “increased bone density” and the like as used interchangeably herein are intended to encompass an increase in the amount of bone due to increased bone deposition and/or decreased resorption in a subject. These terms refer to an overall bone mass increase in an individual or to a localized increase in bone density (e.g., an increase in bone mass at a specific site such as the hip or spine). An increase in bone density can be determined using a variety of techniques as discussed herein.

The term “bisphosphonate” as used herein is generally meant to refer to compounds in which the phosphate-oxygen-phosphate bond (P—O—P) of pyrophosphate is altered so that the oxygen is replaced with a carbon atom (P—C—P). One distinct feature of bisphosphonates is that this substitution produces a compound is not efficiently metabolized by the normal enzymes that break down pyrophosphates. Bisphosphonates have the ability to increase and/or maintain bone mass in an individual upon administration of the bisphosphonate to the individual. Use of the term “bisphosphonate” is not meant to be limiting to use of, or formulations comprising, only one of these selected compounds. Furthermore, reference to a selected specific compound, e.g., reference to “alendronate” or “risedronate” is understood to be only exemplary of the drugs suitable for delivery according to the methods of the invention, and is not meant to be limiting in any way to a specific compound.

The terms “formulation” and “drug formulation” as used herein are intended to encompass any formulation comprising one or more bisphosphonates. The term “formulation” thus encompasses any formulation containing a bisphosphonate. The term “formulation” is also intended to encompass formulations comprising multiple bisphosphonates, and is not meant to be limiting to use of, or formulations comprising, only one of these selected compounds. For example, a formulation may have two or more bisphosphonates, and thus act via a similar mechanism. In another example, a bisphosphonate can be combined with an additional ingredient that increases or maintains bone mass, e.g., calcitonin or a selective estrogen receptor modulator (SERM). Furthermore, reference to a bisphosphonate in a formulation, e.g., reference to alendronate, is understood to be only exemplary of the drugs suitable for delivery according to the methods of the invention, and is not meant to be limiting in any way. The term “formulation” as used herein is also intended to encompass a formulation having at least one bisphosphonate and another active agent, i.e. an active agent that mediates a separate biological response (e.g., a corticosteroid).

The term “corticosteroid” refers to a class of therapeutic agents useful in treatment of inflammatory conditions, including those resulting from infection, transplant rejection and autoimmune disorders. Corticosteroids include those that are naturally occurring, synthetic, or semi-synthetic in origin, and are characterized by the presence of a steroid nucleus of four fused rings, for example, as found in cholesterol, dihydroxycholesterol, stigmasterol, and lanosterol structures. Corticosteroid drugs include cortisone, cortisol, hydrocortisone (11.beta.17-dihydroxy-21-(phosphonooxy)-pregn-4-ene3,20-dione disodium), dihydroxycortisone, dexamethasone (21-(acetyloxy)-9-fluoro-11.beta., 17-dihydroxy-16.alpha.-methylpregna-1,4-diene-3,20-dione), and highly derivatized steroid drugs such as beconase (beclomethasone dipropionate, which is 9-chloro-11.beta.,17,21, trihydroxy-16.beta.-methylpregna-1,4 diene-3,20-dione 17,21-dipropionate). Other examples of corticosteroids include flunisolide, prednisone, prednisolone, methylprednisolone, triamcinolone, deflazacort and betamethasone.

The term “subject” is meant any subject, generally a mammal (e.g., human, canine, feline, equine, bovine, etc.), in which management of bone mass is desired.

The term “systemic delivery” is meant to encompass all parenteral routes of delivery which permit drug to enter into the systemic circulation, e.g., intravenous, intra-arterial, intramuscular, subcutaneous, intra-adipose tissue, intra-lymphatic, etc.

The term “therapeutically effective amount” is meant an amount of a therapeutic agent, or a rate of delivery of a therapeutic agent, effective to facilitate a desired therapeutic effect. The desired therapeutic effect (e.g., an increase in bone density and/or maintenance of present bone mass) will vary according to the severity of the condition to be treated, the formulation to be administered, and a variety of other factors that are appreciated by those of ordinary skill in the art.

“Delivery site” as used herein is meant to refer to an area of the body to which drug is released from the dosage form for entry into the body. Exemplary delivery sites compatible with systemic delivery of drug include, but are not necessarily limited to, subcutaneous, intravenous, intra-arterial, intramuscular, intra-adipose tissue, and intra-lymphatic sites.

The term “implantation site” is used to refer to a site within the body of a subject at which a dosage form is introduced and positioned.

“Drug delivery device” as used herein is meant to refer to any implantable device suitable for delivering the formulations for bone density management according to the invention and thus encompasses any implantable device with any mechanism of action including diffusive, erodible, or convective systems, e.g., osmotic pumps, biodegradable implants, electrodiffusion systems, electroosmosis systems, electrochemical systems, vapor pressure pumps, electrolytic pumps, effervescent pumps, piezoelectric pumps, erosion-based systems, or electromechanical systems. The term “drug delivery device” generally refers to any means for containing and releasing a drug wherein the drug is released into a subject. Drug delivery devices are split into five major groups: inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes two sub-groups: injectable and non-injectable devices. Non-injectable devices are generally referred to as “implants” or “non-injectable implants” and include e.g., pumps and solid biodegradable polymers. Injectable devices are split into bolus injections, that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc.

“Patterned” or “temporal” as used in the context of drug delivery is meant delivery of drug in a pattern, generally a substantially regular pattern, over a pre-selected period of time (e.g., other than a period associated with, for example a bolus administration (e.g., as in a bolus injection or in bolus oral delivery). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not the environment of use.

By “substantially continuous” as used in, for example, the context of “substantially continuous subcutaneous infusion” or “substantially continuous delivery” is meant to refer to delivery of drug (e.g., a bisphosphonate) in a manner that is substantially uninterrupted for a pre-selected period of drug delivery (other than a period associated with, for example, a bolus administration, e.g., by injection or by oral administration). Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

The term “sustained release dosage form” is meant to refer to a drug dosage form that is capable of release of a drug formulation (e.g., a bisphosphonate) over a pre-selected period of time rather than at one time as in a bolus administration (e.g., by injection or oral administration). Sustained release dosage forms can include dosage forms capable of controlled release or patterned release of a drug.

The terms “treat”, “treatment” and the like as used herein generally refer to obtaining a desired pharmacologic and/or physiologic effect. The effect may be prophylactic in terms of completely or partially preventing a condition or symptom thereof and/or may be therapeutic in terms of a partial or complete cure for, relief from, or suppression of a disease and/or adverse effects attributable to the disease. “Treatment” as used herein covers any treatment for the increase or maintenance of bone mass in an animal, particularly a human, and includes:

(a) preventing or diminishing loss of bone density in a subject that may be predisposed but is not at the time displaying electable loss;

(b) reducing bone resorption;

(c) increasing bone deposition; and

(d) causing regression and/or amelioration in a subject with a disease or condition associated with decreased levels of bone mass.

Biphosphonates and Formulations

The present invention provides methods for increasing bone mass and reducing fracture risk in a subject by long-term administration of a bisphosphonate. The agent may be any compound with the ability to increase bone mass levels by any mechanism. A formulation of the invention will comprise one or more bisphosphonates, alone or in conjuction with other therapeutics such as calcitonin, estrogen, an SERM, and the like.

The bisphosphonate class of drugs is based on the phosphate-oxygen-phosphate bond (P—O—P) of pyrophosphate (a widely distributed, natural human metabolite that has a strong affinity for bone). Replacing the oxygen with a carbon atom (P—C—P) produces a group of bone-selective drugs that cannot be metabolized by the normal enzymes that break down pyrophosphates. Gatti D and Adami S Drugs Aging 15:285-96 (1999). Exemplary bisphosphonate compounds for use in the present invention include, but are not limited to, those described U.S. Pat. Nos. 6,090,410; 6,008,207; 6,008,206; 5,994,329; 5,958,908; 5,854,227; 5,849,726; 5,804,570; 5,681,590; 5,583,122; 5,574,024; 5,431,920; 5,358,941; 5,356,887; 5,344,825; 5,270,365; 5,237,094; 5,227,506; 5,183,815; 5,070,108; 5,041,428; 4,980,171; 4,963,681; 4,942,157; 4,927,814; 4,922,007; 4,876,248; 4,711,880; 4,621,077; 4,267,108; and 4,054,598. The methods of the invention envision the methods and devices described herein to include the administration of new drugs of the bisphosphonate class.

In addition, the formulations for use in the present invention are intended to include formulations having two or more agents having different mechanisms (e.g., a bisphosphonate and estrogen) can be used in a single formulation. Therapy with a combination of antiresorptive agents has been shown to increase the gains in bone mass observed with single agents, particularly if the two agents have different sites of action in the bone remodeling cycle, e.g., etidronate and estrogen. See Wimalawansa S J. Am J Med 99: 36-42 (1995).

A bisphosphonate can be provided in any of a variety of formulations compatible with parenteral delivery, provided that such formulation is stable (i.e., not subject to degradation to an unacceptable amount at body temperature). The concentration of bisphosphonate in the formulation may vary from about 0.1 wt. % to about 50 or 75 wt. %. The agent can be provided in any form suitable to be carried by the sustained release dosage from and released parenterally for systemic distribution, e.g., solid, semi-solid, gel, liquid, suspension, emulsion, osmotic dosage formulation, diffusion dosage formulation, erodible formulation, etc. Of particular interest is the administration of a bisphosphonate in a form suitable for administration using an implanted pump.

Formulations of the invention comprise a bisphosphonate in a concentration of at least about 0.1 mg/mL, 0.5 mg/mL, 1 mg/mL, 10 mg/mL, 25 mg/mL, 50 mg/mL, 75 mg/mL, 100 mg/mL, 150 mg/mL, 200 mg/mL, 225 mg/mL, 250 mg/mL, 300 mg/mL, 350 mg/mL, 400 mg/mL, 450 mg/mL, 500 mg/mL, or greater. Formulations of the invention comprising bisphosphonate are preferably in solution, e.g., are dissolved in a liquid.

Pharmaceutical grade organic or inorganic carriers and/or diluents suitable for parenteral delivery can be included in the formulations suitable for delivery according to the invention. Such physiologically acceptable carriers are well known in the art. Exemplary liquid carriers for use in accordance with the present invention can be sterile non-aqueous or aqueous solutions which contain no materials other than the active ingredient. The formulations can optionally further comprise a buffer such as sodium phosphate at physiological pH value, physiological saline or both (i.e., phosphate-buffered saline). Suitable aqueous carriers may optionally further comprise more than one buffer salt, as well as other salts (such as sodium and potassium chlorides) and/or other solutes.

In a particular embodiment, the formulations of the invention comprise a pharmaceutically acceptable aqueous carrier. In specific embodiments, the bisphosphonate is present in the formulation in a concentration of from about 0.1 mg/mL, 0.5 mg/mL to about 500 mg/mL, from about 1 mg/mL to about 450 mg/mL, from about 50 mg/mL to about 400 mg/mL, from about 75 mg/mL to about 300 mg/mL, or from about 100 mg/mL to about 250 mg/mL.

Formulations of particular interest for delivery are characterized in that the bisphosphonate is present in a high concentration, as described above. The bisphosphonate may be provided to the subject as a solution, a suspension, and/or a precipitate.

The formulations suitable for administration according to the invention comprise a bisphosphonate and optionally an additional active or inert components that are pharmaceutically acceptable and compatible with the bisphosphonate. Suitable excipients can comprise dextrose, glycerol, alcohol (e.g., ethanol), and the like, and combinations of one or more thereof with vegetable oils, propylene glycol, polyethylene glycol, benzyl alcohol, benzyl benzoate, dimethyl sulfoxide (DMSO), organics, and the like to provide a suitable composition. In addition, if desired, the composition can comprise hydrophobic or aqueous surfactants, dispersing agents, wetting or emulsifying agents, isotonic agents, pH buffering agents, dissolution promoting agents, stabilizers, antiseptic agents and other typical auxiliary additives employed in the formulation of pharmaceutical preparations.

Conjunctive Therapy

In one embodiment, the formulations of the invention include both 1) an agent that is known or suspected of causing bone loss upon chronic use and 2) a bisphosphonate to help suppress or reverse the activity of the first agent. For example, in subjects chronically receiving corticosteroids, the formulation may comprise the corticosteroid and the bisphosphonate. Accordingly, the methods of the invention can be used to treat those subjected to long-term corticosteroid use to help prevent and/or ameliorate bone loss due to the formulations. Corticosteroid formulations suitable for administration are well known in the art and commercially available. For example, dexamethasone acetate, is suitable the treatment of rheumatoid, dermatological, ophthalmic, gastrointestinal, hematologic, neoplastic, allergic conditions and collagen disorders. Hydroxycortisone is available as a potent anti-inflammatory agent for conditions such as osteoarthritis, rheumatoid arthritis, juvenile rheumatoid arthritis, acute and chronic bursitis. For chronic, long term administration, these formulations can be adapted for parenteral dosing and administered to a subject simultaneously with a bisphosphonate.

Such conjuctive formulations may also comprise a bisphosphonate and an antihypertensive, antidepressive, anticonvulsive and the like as will be apparent to one skilled in the art upon reading the present disclosure.

Indications for Administration of Bisphosphanates

In general, administration of a formulation comprising a an agent according to the invention can be used to facilitate management of bone mass and density in individuals with a measured loss of bone density (e.g., as determined by a BMD test) or an individual having any of a wide variety of risk factors, disorders, conditions, or diseases. Conditions associated with bone loss may be caused by either local or systemic unbalanced osteoclast-osteoblast activities.

Specific examples of conditions, diseases, and disorders associated with such a loss of bone mass include osteoporosis, osteopenia, osteogenesis imperfecta, tumor-induced hypercalcemia, pediatric resistant calcemia, recalcitrant reflex sympathetic dystrophy, heterotopic ossification in spinal cord-injured patients, Cushing syndrome, Cystic Fibrosis, and hyperparathyroidism. In addition, inflammatory responses affecting bones that involve macrophages such as rheumatoid arthritis can cause local bone destruction and permanent disability and deformity. Exemplary disorders and conditions are discussed in further detail below.

Osteoporosis

Osteoporosis is the leading disorder associated with loss of bone density and bone mass. When bone loss occurs without symptoms, the subject may not know that he has osteoporosis until their bones become so weak that a sudden strain, bump, or fall causes a fracture or a vertebra to collapse. Collapsed vertebrae may initially be felt or seen in the form of severe back pain, loss of height, or spinal deformities such as kyphosis or stooped posture. These physical changes are indicative of osteoporosis, and the methods and devices of the present invention are useful in an individual displaying such symptoms.

In addition, it is desirable to administer formulation to an individual prior to the onset of physical indications of osteoporosis. The methods and devices of the invention can thus also be used in a patient displaying such risk factors to help curb the disease prior to the presence of physical problems such as fracture and spinal deformities. Certain factors are linked to the development of osteoporosis or contribute to an individual's likelihood of developing the disease. Exemplary risk factors include: 1) gender (women have less bone tissue and lose bone more rapidly than men due to changes involved in menopause); 2) age (risk of osteoporosis, increase with age as bones become less dense and weaker with age); 3) body size (small, thin-boned women are at greater risk); 4) ethnicity (Caucasian and Asian women are at highest risk, while African-American and Latino women have a lower but significant risk); 5) family history (susceptibility to fracture appears to be, in part, hereditary); 6) sex hormone levels (osteoporosis is more prevalent in individuals having amenorrhea, post-menopausal women, and men having low testosterone levels); 7) anorexia; 8) dietary considerations, such as a diet chronically low in calcium and vitamin D; 9) use of certain medications, such as glucocorticoids or some anticonvulsants; 10) an inactive lifestyle or extended bed rest; 11) cigarette smoking; and 12) excessive use of alcohol. In addition, certain disorders such as endocrine diseases e.g., primary hyperparathyroidism or thyrotoxicosis-osteoporosis, or diseases that affect kidney function can also increase the likelihood that an individual will develop osteoporosis. Exemplary methods and apparatus for diagnosing osteoporosis are described in U.S. Pat. Nos. 5,817,020; 5,800,363; 5,749,363; 5,772,596; 5,772,592; and 5,769,074.

Paget's Disease

Paget's disease is a chronic disorder that typically results in enlarged and deformed bones. The excessive breakdown and formation of bone tissue that occurs with Paget's disease can cause bone to weaken, resulting in bone pain, arthritis, deformities, and fractures. Paget's disease may be caused by a “slow virus” infection, present for many years before symptoms appear. There is also a hereditary factor since the disease may appear in more than one family member. Bisphosphonates have been shown to increase bone mass in patients, in slowing disease progression, and in preventing and treating complications arising from this disorder. Trombetti A, Rev Rhum Engl Ed. 66:467-76 (1999).

Tumor-induced Hypercalcemia

Tumor-induced hypercalcemia is the accelerated release of excessive calcium into the blood stream from bone breakage in cancer patients. This condition develops in almost all types of cancer, but most frequently observed in patients with breast cancer, multiple myeloma, and non-small cell lung cancer. Hypercalcemia, one of the most common metabolic complications of cancer, is potentially life-threatening. Another common complication of cancer is invasion of bone by tumor cells that can also cause hypercalcemia due to local production of factors by tumor cells that stimulate bone resorption. Excessive release of calcium from bone causes other physiological disturbances such as polyurea, gastrointestinal problem, and progressive dehydration that causes increased absorption of calcium in the kidney elevating its blood level further. Management of hypercalcemia in cancer patients, therefore requires prevention of bone resorption, and bisphosphonates have been shown to be efficacious in combating this complication. In addition, evidence indicates that bisphosphonates inhibit metastasis of certain cancers to bone, and thus may have important prophylactic benefits as well. Ohnishi T, Jpn J Clin Oncol 30:410-3 (2000).

Prevention of Cancer Progression

Besides their role in preventing the effects of hypercalcemia in cancer patients, bisphopsphonates also have an antimetastatic/antitumor effect. Administration of bisphosphonate to patients suffering from certain forms of cancer (e.g., breast cancer, myeloma, and prostate carcinoma), or to patients at risk for these cancers, may help to ameliorate or prevent spread and/or progression of the disease. See, e.g., Berenson J R et al., Cancer, 91:144-154 (2001).

The bisphosphonates may exhibit their antimetastatic and antitumor effects through an apoptotic and/or antiproliferative effect on osteoclasts, macrophages and tumor cells. Preclinical studies showed that down-regulation of bone metabolism by bisphosphonates is associated with a lower incidence of bone metastases and destruction in animals, whereas activation is correlated with a higher number of metastases. Diel I J et al., Cancer, 88:3080-8 (2000). Two separate studies in a relatively small number of patients treated with clodronate have suggested that this bisphosphonate decreases tumor burden in bone. Mundy G R. Bisphosphonates as anticancer drugs. Expert Opin Investig Drugs. 8:2009-2015 (1999).

Improvements in the survival time have been found in many Phase III studies with bisphosphonates to date, both in the setting of metastatic breast carcinoma and in multiple myeloma. Diel I J, Drugs 59:391-9 (2000). For example, rapid infusion of bisphosphonate has been shown to have efficacy against bone metastases from breast cancer Oura S, et al., Breast Cancer 7:307-310 (2000). In another example, bisphosphonate treatment of painful osseous metastases due to hormone refractory prostate cancer results in a significant pain decrease in 75% of patients. Heidenreich A, et al., J Urol. 165:136-40 (2001).

Pediatric Uses

In recent years, bisphosphonates have been used to treat children acutely for resistant hypercalcemia and chronically for various metabolic bone diseases. The theoretical concerns of possible adverse effects of these drugs on the growing skeleton have not been proven to be true. Clin Pediatr (Phila) 38:687-702 (1999). Although not yet approved by the FDA for use in children, bisphosphonates demonstrate benefits with no serious adverse skeletal effects. Bisphosphonates might be the first agents to provide the pediatrician with an opportunity to treat mineral and bone disorders of childhood, which until recently did not have satisfactory therapy.

Crohn's Disease

Low bone mineral density is a common complication of Crohn's disease and may lead to increased morbidity and mortality because of fractures. Treatment with the bisphosphonate alendronate has been shown to significantly increased BMD in patients with Crohn's disease and was safe and well tolerated. Haderslev K V et al., Gastroenterology 119:639-46 (2000).

Osteogenesis Imperfecta

Osteogenesis imperfecta (OI) is a heterogeneous group of disorders principally affecting type I collagen. Children with the severe forms of the condition suffer recurrent fractures resulting in limb and spine deformities, and restricted ambulation. Recently, cyclical intravenous administration of bisphosphonate has proven of benefit to children with the severe forms of OI. Glorieux F H, J Pediatr Endocrinol Metab Sep. 13, 2000 Suppl 2:989-92 (2000). Bone mineral density increased, and the incidence of fractures decreased. The treatment does not alter fracture healing, growth rate, or growth plate appearances. Dependence on mobility aids is reduced and there is substantial relief of chronic pain and fatigue.

Use of Medications Associated with Bone Loss

A number of different therapeutics, including but not limited to antihypertensives, glucocorticoids, antidepressives, and anticonvulsives, have been shown to cause bone loss in individuals receiving such therapeutics, and in particular in those receiving the therapeutics long term. For example, long-term use of the anticonvulsants phenytoin and carbamazepine has been shown to decrease bone mass in patients, and directly suppress proliferation of osteoblast-like cells. Feldkamp J, Exp Clin Endocrinol Diabetes. 108:37-43 (2000). Recent clinical studies have demonstrated a marked decrease in bone mineral density in women with histories of depression (Schweiger U, Am J Psychiatry 157:118-20 (2000); Michelson D, N Engl J Med 335:1176-81 (1996)), suggesting that chemical inbalances associated with depression and/or antidepressive drugs also affect bone deposition and/or resorption.

In a particular example, glucocorticoid drugs interact with bone metabolism at many levels, but their principal action is to reduce osteoblast number and bone matrix synthesis. Virtually all patients receiving glucocorticoids in doses above 5 mg per day lose bone, the amount lost being dependent on the cumulative steroid dose. Reid I R Baillieres Best Pract Res Clin Endocrinol Metab 14:279-98 (2000). The combination of increasing age and corticosteroid use is associated with a marked increase in the risk of vertebral deformity. Administration of bisphosphonate formulations according to the present invention can be used to reverse this bone loss, and to prevent additional bone loss in subjects continuing therapy.

Direct Measurement of Bone Mass

Bisphosphonates may be administered according to the methods of the invention in individuals at fracture risk for osteoporosis as determined by a bone mass measurement, e.g., a bone mineral density test. There are numerous ways to measure bone mineral density which are painless, noninvasive and safe and are becoming more readily available. The majority of tests measure bone density in spine, hip and/or wrist, the most common sites of fractures due to osteoporosis. An individual's bone mass is compared to two standards, or norms, known as “age matched” and “young normal.” The age-matched reading compares an individual's bone mass to what is expected in someone of the same or similar age, sex and size. The young normal reading compares an individual's bone mass to the optimal peak bone mass of a healthy young adult of the same sex. In general, the lower an individual's bone mass, the higher the risk for fracture.

Generally, devices that can be used for determining bone mass are central machines that measure mass in the hip, spine and total body, and peripheral machines measure mass in the finger, wrist, kneecap, shin bone and heel. Exemplary machines approved for use in determining an individual's bone mass include, but are not limited to, DXA (Dual Energy X-ray Absorptiometry), which measures the spine, hip or total body; pDXA (Peripheral Dual Energy X-ray Absorptiometry), which measures the wrist, heel or finger; SXA (single Energy X-ray Absorptiometry) which measures the wrist or heel; QUS (Quantitative Ultrasound), which uses sound waves to measure mass at the heel, shin bone and kneecap; QCT (Quantitative Computed Tomography) which is most commonly used to measure the spine, but can be used at other sites; pQCT (Peripheral Quantitative Computed Tomograph), which measures the wrist; RA (Radiographic Absorptiometry), which uses an X-ray of the hand and a small metal wedge to calculate bone mass; DPA (Dual Photon Absorptiometry), which measures the spine, hip or total body (used infrequently); and SPA (Single Photon Absorptiometry) which measures the wrist. Bone mass measurements using any of these devices, or mass measurements based on a combination of these devices, can be used to determine the risk of an individual for fracture, and thus the need for administration of a bisphosphonate according to the methods of the invention.

In addition, biochemical tests can be used to identify loss of bone density, either alone or in conjunction with the above-described methods.

Implantation and Delivery Sites

Drug Delivery Devices Generally

The dosage form comprising the formulation can be introduced to a subject by implantation at any suitable site using methods and devices well known in the art. Implantation sites include, but are not necessarily limited to a subdermal, subcutaneous, intramuscular, or other suitable site within a subject's body. Subcutaneous implantation sites are preferred because of convenience in implantation and, if necessary, removal of the drug dosage form. In some embodiments, the implantation site is at or near the delivery site (e.g., the delivery site is not distant from the implantation site). In some embodiments, the delivery site is distant form the implantation site. Delivery of drug from a dosage form at an implantation site that is distant from a delivery site can be accomplished by providing the drug delivery device with a catheter, as described in more detail below.

Delivery sites compatible with systemic delivery include, but are not necessarily limited to, subcutaneous, intravenous, intra-arterial, intramuscular, intra-adipose tissue, intra-lymphatic and sublingual sites. Subcutaneous delivery sites are of particular interest in the present application. Exemplary subcutaneous delivery sites include external subcutaneous sites (e.g., under the skin of the arm, shoulder, neck, back, or leg) and internal subcutaneous sites within a body cavity (e.g., within the mouth). In addition, the delivery site can be the desired site of action (e.g., specific vessels at or near the heart or brain, etc.).

A drug can be administered using any of a number of delivery systems, including sustained release devices. In some embodiments, the drug delivery system will comprise a catheter operably attached to a sustained release drug delivery device. A proximal end of the catheter is operably attached to a sustained release drug delivery device; and a distal end of the catheter releases drug close to the intended site of action. In other embodiments, the drug delivery device is a depot.

In general, the drug delivery devices suitable for use in the invention comprise a drug reservoir for retaining a drug formulation or alternatively some substrate or matrix which can retain drug (e.g., a polymer; a viscous non-polymer compound, e.g., as described in U.S. Pat. No. 5,747,058 and U.S. application Ser. No. 09/385,107; a binding solid, etc).

The delivery device is generally adapted for delivery of a drug over extended periods of time. Such delivery devices may be adapted for administration of a drug for several hours (e.g. greater than 12 hours), days (e.g. greater than 7 days), weeks (e.g. greater than 4 weeks) months (e.g. greater than three months) or years.

Release of drug from the device can be accomplished in any of a variety of ways according to methods well known in the art, e.g., by incorporation of drug into a polymer that provides for sustained diffusion of drug from within the polymer, incorporation of drug in a biodegradable polymer, providing for delivery of drug from an osmotically-driven device, etc.

The drug reservoir or other means for holding or containing the drug must also be of such material as to avoid unintended reactions with the active agent formulation, and is preferably biocompatible. Suitable materials for the reservoir or drug holding means may comprise a non-reactive polymer or a biocompatible metal or alloy. Exemplary polymers include, but are not necessarily limited to, silicone, polyurethane, polyether urethane, polyether urethane urea, polyamide, polyacetal, polyester, polytetrafluoroethylene (PTFE or “Teflon™”), polyanhydrides, cyclodextrans, polylactic-glycolic acid, polycaprolactone, polyorthoesters, n-vinyl alcohol, polyglycolic acid, polylactic acid and copolymers thereof.

Drug release devices suitable for use in the invention may be based on any of a variety of modes of operation. For example, the drug release device can be based upon a diffusive system, a convective system, or an erodible system (e.g., an erosion-based system). For example, the drug release device can be an osmotic pump, an electroosmotic pump, an electrochemical pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

A drug delivery device of the invention may release drug in a range of rates of from about 0.01 microgram/hr to about 200 microgram/hr, and which can be delivered at a volume rate of from about 0.01 microliter/day to about 100 microliter/day, e.g. 0.2 microliter/day to about 5 microliter/day. In particular embodiments, the volume/time delivery rate is substantially constant (e.g., delivery is generally at a rate of about 5% to 10% of the cited volume over the cited time period.

The drug delivery device can be implanted at any suitable implantation site using methods and devices well known in the art. An implantation site is a site within the body of a subject at which a drug delivery device is introduced and positioned. Implantation sites include subdermal, subcutaneous, intramuscular etc.

Pumps

Drug release devices based upon a mechanical or electromechanical infusion pumps can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. The DUROS™ osmotic pump is particularly suitable (see, e.g., WO 97/27840 and U.S. Pat. Nos. 5,985,305 and 5,728,396, hereby incorporated by reference).

Depots

The drug delivery device can be a depot. Depots are injectable drug delivery devices that may comprise polymeric and/or non-polymeric materials, and are provided in liquid, or semi-solid forms that release drug over time.

Exemplary non-polymeric materials useful in making a depot dosage form include, but are not necessarily limited to, those described in U.S. Pat. Nos. 6,051,558; 5,747,058; and 5,968,542, e.g. a non-polymeric material having a viscosity of at least 5000 cP at 37° C., for example, SAIB.

Suitable polymeric materials include, but are not limited to, polyanhydrides; polyesters such as polyglycolides and polylactide-co-glycolides; polyamino acids such as polylysine; polymers and co-polymers of polyethylene oxide; acrylic terminated polyethylene oxide; polyamides; polycaprolactone, polyurethanes; polyorthoesters; polyacrylonitriles; and polyphosphazenes. See, e.g., U.S. Pat. Nos. 4,891,225; 4,906,474; 4,767,628; and 4,530,840. Degradable materials of biological origin include, but are not limited to, cross-linked gelatin; and hyaluronic acid (e.g., U.S. Pat. No. 4,767,628). A depot may also be provided in the form of a biodegradable hydrogel. See, e.g., U.S. Pat. No. 5,149,543. Depots also include materials that exist in one physical state outside the body, and assume a different physical state when introduced into the body. Examples include liquid materials that form solids when placed within an individual, with or without addition of a catalyst. See, e.g., U.S. Pat. No. 4,938,763. A number of factors well known to those familiar with the art will have an effect on depot release kinetics and should be considered in designing an effective formulation. For example a smaller injection will give a depot with a larger surface-to-volume ratio than a depot resulting from a larger injection. For example, one formulation tested in vitro may have a burst of over 50% when evaluated at a 100 mg depot size and less than 25% when evaluated at a 1000 mg depot size.

Alternatively, the drug delivery device can be a dispersion system, e.g., a suspension or an emulsion. Suspensions are solid particles ranging in size from a few nanometers to hundreds of micrometers, dispersed in a liquid medium using a suspending agent. Solid particles include microspheres, microcapsules, and nanospheres. Emulsions are dispersions of one liquid in another, stabilized by an interfacial film of emulsifiers such as surfactants and lipids. Emulsion formulations include water in oil and oil in water emulsions, multiple emulsions, microemulsions, microdroplets, and liposome emulsions.

Delivery of Bisphosphonates

In general, the formulation comprising a bisphosphonate is delivered at a volume rate that is compatible with the delivery site, and at a dose that is therapeutically effective in increase in and/or maintenance of bone mass in an individual.

Subjects suffering from or susceptible to decreased bone density, spinal deformities, and fracture can receive prophylactic and/or therapeutic amounts of a bisphosphonate according to the methods of the invention for any desired period of time. As conditions associated with decreased bone mass are generally chronic, long-term administration is preferred, and the administration of a bisphosphonate according to the invention can be sustained for several days (e.g., 2 to 5 days or more), to several weeks, months or years. Typically, delivery can be continued for a period ranging from about 1 week to about 1 month or about 12 months or more. The bisphosphonate may be administered to an individual for a period of, for example, from about 20 days, from about 7 days or more, from about 10 days or more, from about 100 days or more, from about 1 week to about 4 weeks, from about 1 month to about 24 months, from about 2 months to about 12 months, from about 3 months to about 9 months, from about 1 month or more, from about 2 months or more, or from about 6 months or more; or other ranges of time, including incremental ranges, within these ranges, as needed.

Preferably, delivery of bisphosphonate is in a patterned fashion, more preferably in a substantially continuous fashion, e.g., substantially uninterrupted for a pre-selected period of drug delivery, and more preferably at a substantially constant, pre-selected rate or range of rates (e.g., amount of agent per unit time, or volume of drug formulation for a unit time). The agent can be delivered at a low volume rate of, for example, from about 0.001 μl/day or 0.04 μl/day to about 1 ml/day, usually from about 0.001 ml/day (1 μl/day) to at least about 500 μl/day or about 1 ml/day (i.e., from about 0.04 μl/hr to about 21 μl/hr to about 42 μl/hr), from about 2 μl/day to about 250 μl/day to 500 μl/day, from about 4 μl/day to about 100 μl/day, from about 5 μl/day to about 50 μl/day to 250 μl/day.

Administration of a formulation by parenteral delivery according to the invention is particularly preferred where other forms of administration are undesirable, e.g., the subject has difficulty with compliance with the desired dosage regime. In particular, sustained release dosage forms are convenient to the subject for long-term drug administration and can allow drug therapy to be conducted on an out-patient basis where the patient's health allows such. Implantable dosage forms, e.g., pumps or depot injections, have an added benefit in that they reduce the risk of infection associated with external pumps or other methods that require repeated breaking of the skin and/or maintenance of a port for administration.

Delivery of drug to a subcutaneous site at a low volume rate is a particularly preferred embodiment of the invention. In general, low volume rate drug delivery avoids accumulation of drug at the delivery site (e.g., depot or pooling effect) by providing for a rate of administration that is less than, the same as, or only very slightly greater than the rate of removal of drug from the delivery site (e.g., by absorption of drug in tissues at the site, movement of drug away from the site by flow of blood or other bodily fluids, etc.). Thus, in addition to providing an implantable system for long-term delivery of bisphosphonates (e.g., alendronate or risedronate), the present invention also provides a method for treating bone loss by balancing the rates of drug absorption and drug delivery to accomplish administration of a therapeutically effective amount of drug, while avoiding accumulation of drug at the delivery site.

In one embodiment, a sustained release dosage form provides for substantially continuous, subcutaneous delivery of drug at a preselected rate. For example, for subcutaneous delivery of a bisphosphonate, the agent can be delivered at a rate of from about 0.01 μg/hr to about 1000 μg/hr, usually from about 10 μg/hr to about 750 μg/hr, and typically between about 5 μg/hr to about 500 μg/hr. In a specific exemplary embodiment, a bisphosphonate is delivered at a rate of from about 0.01 μg/hr, 0.1 μg/hr, 0.25 μg/hr, 1 μg/hr, 25 μg/hr, 50 μg/hr, 100 μg/hr, 250 μg/hr, 500 μg/hr, 750 μg/hr, and generally up to about 1000 μg/hr. In another exemplary embodiment, the bisphosphonate is delivered at a rate of from about 1 μg/hr to about 1000 μg/hr, typically between about 10 μg/hr to about 500 μg/hr. Appropriate amounts of bisphosphonate can be readily determined by the ordinarily skilled artisan based upon, for example, the relative potency of these drugs. The actual dose of drug delivered will vary with a variety of factors such as the potency and other properties of the selected drug used (e.g., lipophilicity, etc.).

Dosage Forms for Use in the Invention

Any of a variety of parenteral dosage forms can be used in the present invention to accomplish delivery of a formulation according to the methods of the present invention. Drug delivery methods and dosage forms that may be suitable for use with the present invention can take advantage of any of a variety of drug release mechanisms. In general, the drug release methods or dosage forms suitable for use in the invention are capable of retaining a quantity of drug formulation (e.g., contained in a drug reservoir or solubilized, suspended or integrated into a vehicle, substrate or matrix such as a polymer, binding solid, etc.) sufficient for treatment for a pre-selected period. In general the dosage forms for use with the present invention should be capable of sustained release of the formulation. Exemplary dosage forms include drug delivery devices (e.g., drug pumps), bioerodable implants, sustained release injectables (e.g., injectable high viscous formulations, gels including hydrogels such as collagen hydrogels), microparticulate suspensions, microsphere suspensions, liposome formulations, micelle formulations, oil suspensions (including emulsions), and encapsulated particulate suspensions. Drug delivery dosage forms that may be suitable for use with the present invention are described in Encyclopedia of Controlled Drug Delivery (1999), Edith Mathiowitz (Ed.), John Wiley & Sons, Inc.

Drug delivery devices suitable for use in accordance with the invention can be selected from any of a variety of implantable drug delivery systems known in the art. In a particular embodiment, the dosage form comprises a controlled drug delivery device and, in one embodiment, further comprises a drug delivery catheter for delivery of a drug at a distance from the implantation site. In specific embodiments, the delivery device is one that is adapted for delivery of a formulation over extended periods of time. Such delivery devices may be adapted for administration of a formulation for several days (e.g., 2 to 5 days or more, from about 100 days or more), to several months or years. In some of these embodiments, the device is adapted for delivery for a period ranging from about 1 month to about 12 months or more. The drug delivery device may be one that is adapted to administer a formulation for a period of time that provides increased bone density and/or decreased bone loss for an extended period of time. Generally, bisphosphonate is administered to an individual for at least several days to at least a week, but more preferably it is administered for a longer period of time, for example from about from about 10 days to about 30 days or more, from about 20 days to about 100 days or more; from about 2 week to about 4 weeks, from about 1 month to about 24 months, from about 2 months to about 12 months, from about 3 months to about 9 months, from about 1 month or more, from about 2 months or more, or from about 6 months or more; or other ranges of time, including incremental ranges, within these ranges, as needed. In these embodiments, high-concentration formulations of bisphosphonates described herein are of particular interest for use in the invention. Release of drug from the dosage form, particularly controlled release of drug, can be accomplished in any of a variety of ways according to methods well known in the art, e.g., by solubilization or suspension of drug in a vehicle or incorporation of drug into a polymer that provides for substantially controlled diffusion of drug from within the polymer, incorporation of drug in a biodegradable polymer, providing for delivery of drug from an osmotically-driven device, etc. Where the drug delivery device comprises a drug delivery catheter, drug can be delivered through the drug delivery catheter to the delivery site as a result of capillary action, as a result of pressure generated from the drug device, by diffusion, by electrodiffusion or by electroosmosis through the device and/or the catheter.

In general, the dosage form must be capable of carrying the drug formulation in such quantities and concentration as therapeutically required for treatment over the pre-selected period, and must provide sufficient protection to the formulation from degradation by body processes for the duration of treatment. For example, the dosage form can be surrounded by an exterior made of a material that has properties to protect against degradation from metabolic processes and the risk of, e.g., leakage, cracking, breakage, or distortion. This can prevent expelling of the dosage form contents in an uncontrolled manner under stresses it would be subjected to during use, e.g., due to physical forces exerted upon the drug release device as a result of movement by the subject or for example, in convective drug delivery devices, physical forces associated with pressure generated within the reservoir. The drug reservoir or other means for holding or containing the drug must also be of such material as to avoid unintended reactions with the active agent formulation, and is preferably biocompatible (e.g., where the dosage form is implanted, it is substantially non-reactive with respect to a subject's body or body fluids).

Suitable materials for the reservoir or drug holding means for use in the delivery devices of the invention are well known in the art. For example, the reservoir material may comprise a non-reactive polymer or a biocompatible metal or alloy. Suitable polymers include, but are not necessarily limited to, acrylonitrile polymers such as acrylonitrile-butadiene-styrene polymer, and the like; halogenated polymers such as polytetrafluoroethylene, polyurethane, polychlorotrifluoroethylene, copolymer tetrafluoroethylene and hexafluoropropylene; polyethylene vinylacetate (EVA), polyimide; polysulfone; polycarbonate; polyethylene; polypropylene; polyvinylchloride-acrylic copolymer; polycarbonate-acrylonitrile-butadiene-styrene; polystyrene; cellulosic polymers; and the like. Further exemplary polymers are described in The Handbook of Common Polymers, Scott and Roff, CRC Press, Cleveland Rubber Co., Cleveland, Ohio.

Metallic materials suitable for use in the reservoir of the drug delivery devices include stainless steel, titanium, platinum, tantalum, gold and their alloys; gold-plated ferrous alloys; platinum-plated titanium, stainless steel, tantalum, gold and their alloys as well as other ferrous alloys; cobalt-chromium alloys; and titanium nitride-coated stainless steel, titanium, platinum, tantalum, gold, and their alloys.

Exemplary materials for use in polymeric matrices include, but are not necessarily limited to, biocompatible polymers, including biostable polymers and biodegradable polymers. Exemplary biostable polymers include, but are not necessarily limited to silicone, polyurethane, polyether urethane, polyether urethane urea, polyamide, polyacetal, polyester, poly ethylene-chlorotrifluoroethylene, polytetrafluoroethylene (PTFE or “Teflon™”), styrene butadiene rubber, polyethylene, polypropylene, polyphenylene oxide-polystyrene, poly-a-chloro-p-xylene, polymethylpentene, polysulfone and other related biostable polymers. Exemplary biodegradable polymers include, but are not necessarily limited to, polyanhydrides, cyclodestrans, polylactic-glycolic acid, polyorthoesters, n-vinyl alcohol, polyethylene oxide/polyethylene terephthalate, polyglycolic acid, polylactic acid, sucrose acetate isobutyrate, and other related bioabsorbable polymers.

Where the drug formulation is stored in a reservoir comprising metal or a metal alloy, particularly titanium or a titanium alloy having greater than 60%, often greater than 85% titanium is preferred for the most size-critical applications, for high payload capability and for long duration applications and for those applications where the formulation is sensitive to body chemistry at the implantation site or where the body is sensitive to the formulation. Most preferably, the drug delivery devices are designed for storage with drug at room temperature or higher.

Drug release devices suitable for use in the invention may be based on any of a variety of modes of operation. For example, the drug release device can be based upon a diffusive system, a convective system, or an erodible system (e.g., an erosion-based system). For example, the drug release device can be an osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump, can also be suitable for use with the present invention. Examples of such devices include those described in, for example, U.S. Pat. Nos. 4,692,147; 4,360,019; 4,487,603; 4,360,019; 4,725,852, and the like. In general, the present methods of drug delivery can be accomplished using any of a variety of refillable, non-exchangeable pump systems. Pumps and other convective systems are generally preferred due to their generally more consistent, controlled release over time. Osmotic pumps are particularly preferred due to their combined advantages of more consistent controlled release and relatively small size. Exemplary osmotically-driven devices suitable for use in the invention include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; 5,985,305; and the like.

In one embodiment, the drug release device is a controlled drug release device in the form of an osmotically-driven device. Preferred osmotically-driven drug release systems are those that can provide for release of agent in a range of rates of from about 0.01 μg/hr to about 1000 μg/hr, and which can be delivered at a volume rate range of, for example, from about 0.001 μl/day to about 100 μl/day (i.e., from about 0.0004 μl/hr to about 4 μl/hr), from about 0.04 μl/day to about 10 μl/day, from about 0.2 μl/day to about 5 μl/day, from about 0.5 μl/day to about 1 μl/day. In general, in the present invention, the drug release system is selected to provide for delivery of a bisphosphonate at a rate of from about 0.001 ml/day (1 μl/day) to at least about 500 μl/day or about 1 ml/day (i.e., from about 0.04 μl/hr to about 21 μl/hr to about 42 μl/hr), from about 2 μl/day to about 250 μl/day to 500 μl/day, from about 4 μl/day to about 100 μl/day, from about 5 μl/day to about 50 μl/day to 250 μl/day.

In an embodiment, the sustained release dosage form is a depot-type injectable, see e.g., U.S. Pat. Nos. 6,183,781; 6,174,547; 6,156,331; 6,143,314; 6,130,200; 6,120,789; 6,051,558; 5,989,463; 5,968,542; 5,912,015; 5,747,058; 5,702,716; 5,654,008; and 5,650,173.

In one embodiment of particular interest, the volume/time delivery rate is substantially constant (e.g., delivery is generally at a rate ± about 5% to 10% of the cited volume over the cited time period). In one embodiment, the drug release device is a continuous drug release device in the form of an osmotically-driven device. Preferred osmotically-driven drug release systems are those that can provide for release of drug in a range of rates of from about 0.1 μg/hr to about 1000 μg/hr, and which can be delivered at a volume rate of from about 0.25 μl/day to about 100 μl/day (i.e., from about 0.0004 μl/hr to about 4 μl/hr), from about 0.04 μl/day to about 10 μl/day, and can be from about 0.2 μl/day to about 5 μl/day, or from about 0.5 μl/day to about 1 μl/day. In one embodiment, the volume/time delivery rate is substantially constant (e.g., delivery is generally at a rate ± about 5% to 10% of the cited volume over the cited time period).

Delivery of a Formulation Using a Drug Delivery Device Comprising a Drug Delivery Catheter

In some embodiments wherein a drug delivery device is used, it may be desirable to provide a drug delivery catheter with the drug delivery device, e.g., where the implantation site and the desired delivery site are not the same or adjacent. The drug delivery catheter is generally a substantially hollow elongate member having a first end (or “proximal” end) associated with the drug release device of the drug delivery device, and a second end (or “distal” end) for delivery of the drug-comprising formulation to a desired delivery site. Where a drug delivery catheter is used, a first end of the drug delivery catheter is associated with or attached to the drug delivery device so that the lumen of the drug delivery catheter is in communication with the drug reservoir in the drug delivery device, so that a formulation contained in a drug reservoir can move into the drug delivery catheter, and out a delivery outlet of the catheter which is positioned at the desired delivery site.

The body of the catheter defines a lumen, which lumen is to have a diameter compatible with providing leak-proof delivery of drug formulation from the drug delivery device. Where the drug delivery device dispenses drug by convection, the size of the catheter lumen leading from the reservoir of the drug release system can be designed as described by Theeuwes (1975) J. Pharm. Sci. 64:1987-91.

The body of the catheter can be of any of a variety of dimensions and geometries (e.g., curved, substantially straight, tapered, etc.) that can be selected according to their suitability for the intended site for drug delivery. The distal end of the drug delivery catheter can provide a distinct opening for delivery of drug, or as a series of openings.

The drug delivery catheter may be produced from any of a variety of suitable materials, and may be manufactured from the same or different material as the reservoir of the drug release device. Impermeable materials suitable for use in production of the controlled drug release device as described above are generally suitable for use in the production of the drug delivery catheter. Exemplary materials from which the drug delivery catheter can be manufactured include, but are not necessarily limited to, polymers; metals; glasses; polyolefins (high mass polyethylene (HDPE), low mass polyethylene (LDPE), linear low mass polyethylene (LLDPE), polypropylene (PP), and the like); nylons; polyethylene terephtholate; silicones; urethanes; liquid crystal polymers; PEBAX®; HYTREL®; TEFLON®; perflouroethylene (PFE) perflouroalkoxy resins (PFA); poly(methyl methacrylate) (PMMA); multilaminates of polymer, metals, and/or glass; nitinol; and the like.

The drug delivery catheter can comprise additional materials or agents (e.g., coatings on the external or internal catheter body surface(s)) to facilitate placement of the drug delivery catheter and/or to provide other desirable characteristics to the catheter. For example, the drug delivery catheter inner and/or outer walls can be coated with silver or otherwise coated or treated with antimicrobial agents, thus further reducing the risk of infection at the site of implantation and drug delivery.

In one embodiment, the drug delivery catheter is primed with a drug-comprising formulation, e.g., is substantially pre-filled with drug prior to implantation. Priming of the drug delivery catheter reduces delivery start-up time, i.e., time related to movement of the drug from the drug delivery device to the distal end of the drug delivery catheter. This feature is particularly advantageous in the present invention where the drug release device of the drug delivery device releases a bisphosphonate at relatively low flow rates.

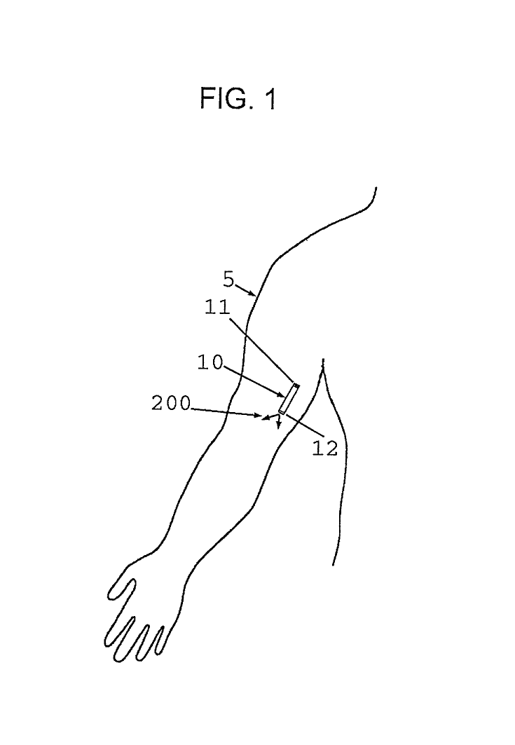

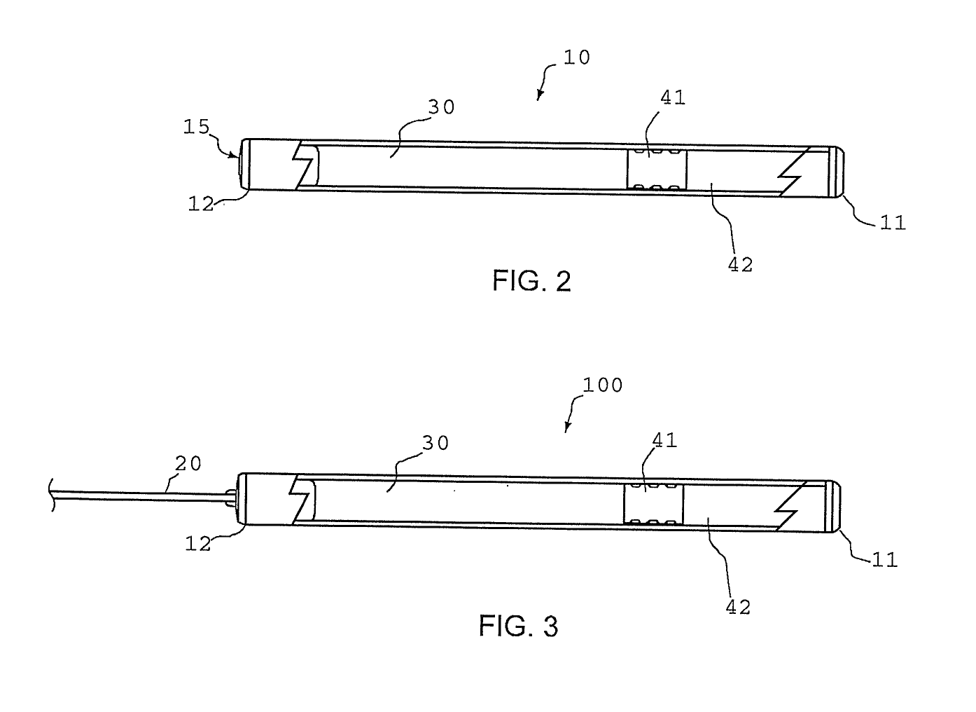

FIG. 1 illustrates one embodiment of the invention, wherein a formulation is delivered from an implanted drug delivery device that provides for sustained release of a formulation from a drug reservoir to a subcutaneous site. In this example, the drug delivery device 10 is implanted at a subcutaneous site in the patient's arm 5. Flow of drug from the device's drug reservoir and to the subcutaneous site is illustrated by arrows 200. FIG. 2 provides a perspective view of the exemplary drug delivery device 10 implanted in FIG. 1. The drug delivery device 10 comprises proximal and distal ends 11 and 12, with the distal end defining an orifice 15 through which drug exits the drug reservoir 30 for delivery to the subcutaneous site. In the exemplary device 10, controlled release of drug from the reservoir 30 is provided by an osmotic engine comprising a piston 41 and a chamber comprising an osmotic engine 42.

As shown in the cut-away of the drug delivery device in FIG. 3, the drug delivery system 100 comprises a drug delivery device 10 and a drug delivery catheter 20. The walls of the drug delivery catheter define a lumen, and the drug delivery catheter is associated with the drug delivery device 10 so that a drug delivery pathway is provided from the drug reservoir 30, through the orifice, and out the distal end 12 of the drug delivery device. The catheter 20 can be positioned for systemic delivery of drug, for example, subcutaneously.

Methods for implanting or otherwise positioning the dosage forms of the invention into the body are well known in the art. In general, placement of parenteral dosage forms will be accomplished using methods and tools that are well known in the art, and performed under aseptic conditions with at least some local or general anesthesia administered to the subject. Removal and/or replacement of the dosage forms, if necessary, can also be accomplished using tools and methods that are readily available.

### EXAMPLES

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g., amounts, temperature, concentrations, etc.) but some experimental errors and deviations should be allowed for. Unless otherwise indicated, parts are parts by weight, molecular weight is average molecular weight, temperature is in degrees centigrade, and pressure is at or near atmospheric.

### Example 1

Administration of Pamidronate

A test was performed to evaluate the local response to pamidronate sodium following subcutaneous injection into a rabbit model.

The pamidronate sodium was reconstituted with 10 mL of phosphate buffered saline (PBS) to achieve a concentration of 9 mg/mL. The 9 mg/mL. solution was then diluted to achieve a concentration of 0.9 mg/mL.

On the day of the test, the fur on the back of each animal was removed on both sides of the spinal column with electric clippers. A 0.1 mL portion of two concentrations of the sample solution was injected subcutaneously at each of three sites along the spinal column of each of two rabbits. A 0.1 mL portion of PBS was injected subcutaneously at three sites along the other side of the spinal column of the same two rabbits. The injection sites were observed for erythema, eschar formation, edema, and necrosis. Scoring was at 24 hours, 48 hours, and 72 hours. All of the animals were observed daily for signs of ill health. The animals remained healthy throughout the test period.

The injection sites were examined and scored for any tissue reactions, with results scored according to Table 1.

TABLE 1
EVALUATION OF SKIN REACTIONS¹

| | Score |
|---|---|
| Erythema and Eschar Formation | |
| No erythema | 0 |
| Very slight erythema | 1 |
| Well-defined erythema | 2 |
| Moderate to severe erythema | 3 |
| Severe erythema (beer-redness) to slight | 4 |
| eschar formation (injuries in depth) | |
| Edema Formation² | |
| No edema | 0 |
| Very slight edema (barely perceptible) | 1 |
| Slight edema (edges of area well defined | 2 |
| by definite raising) | |
| Moderate edema (raised about 1 mm) | 3 |
| Sever edema (raised more than 1 mm | 4 |
| and extending beyond area of exposure) | |

¹From USP 24, January 1, 2000. p. 1835
²Excludes non-inflammatory (mechanical) edema from the bunk or extraction fluid.

The average erythema and edema scores for each test article and controls are determined at each scoring interval (24, 48, and 72 hours) for each rabbit.

TABLE 2
Skin Reaction Scores- Sodium Chloride for Injection Sample and Blank

| | | Test Sites | | | Test Sites | | | PBS Control Sites | | | | | |
|---|---|---|---|---|---|---|---|---|---|---|---|---|---|
| Time | Reaction | 1 | 2 | 3 | 4 | 5 | 6 | 7 | 8 | 9 | 10 | 11 | 12 |

Rabbit #26808.M

| | | | | | | | | | | | | | |
|---|---|---|---|---|---|---|---|---|---|---|---|---|---|
| 24 hr | Erythema | 1 | 0 | 0 | 0 | 0 | 0 | 0 | 0 | 0 | 0 | 0 | 0 |
| | Edema | 0 | 0 | 0 | 0 | 0 | 0 | 0 | 0 | 0 | 0 | 0 | 0 |
| 48 hr | Erythema | 0 | 0 | 0 | 0 | 0 | 0 | 0 | 0 | 0 | 0 | 0 | 0 |
| | Edema | 0 | 0 | 0 | 0 | 0 | 0 | 0 | 0 | 0 | 0 | 0 | 0 |
| 72 hr | Erythema | 0 | 0 | 0 | 0 | 0 | 0 | 0 | 0 | 0 | 0 | 0 | 0 |
| | Edema | 0 | 0 | 0 | 0 | 0 | 0 | 0 | 0 | 0 | 0 | 0 | 0 |

Rabbit #26853.M

| | | | | | | | | | | | | | |
|---|---|---|---|---|---|---|---|---|---|---|---|---|---|
| 24 hr | Erythema | 0 | 1 | 0 | 0 | 0 | 0 | 0 | 0 | 0 | 0 | 0 | 0 |
| | Edema | 0 | 0 | 0 | 0 | 0 | 0 | 0 | 0 | 0 | 0 | 0 | 0 |
| 48 hr | Erythema | 0 | 1 | 0 | 0 | 0 | 0 | 0 | 0 | 0 | 0 | 0 | 0 |
| | Edema | 0 | 0 | 0 | 0 | 0 | 0 | 0 | 0 | 0 | 0 | 0 | 0 |
| 72 hr | Erythema | 0 | 1 | 0 | 0 | 0 | 0 | 0 | 0 | 0 | 0 | 0 | 0 |
| | Edema | 0 | 0 | 0 | 0 | 0 | 0 | 0 | 0 | 0 | 0 | 0 | 0 |

Slight erythema was noted at one of the 9 mg/mL sites at 24 hours for both animals, and at 48 and 72 hours for one 9 mg/mL site on one rabbit. There was no evidence of erythema or edema at any of the 0.9 mg/mL or control sites.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, process, process step or steps, to the objective, spirit and scope of the present invention. All such modifications are intended to be within the scope of the claims appended hereto.

1. A method for increasing bone density in a subject, the method comprising: parenterally implanting in said subject a sustained release dosage form, said sustained release dosage form comprising a drug delivery device and a bone density-modulating drug, and administering said drug from said dosage form into said subject, for a period of at least 24 hours, in an dose sufficient to cause a measurable increase in bone density.

2. The method of claim 1, wherein said drug is a bisphosphonate.

3. The method of claim 2, wherein said drug delivery device is selected from the group consisting of: a pump, a bioerodable implant, and a depot.

4. The method of claim 3, wherein said bisphosphonate is administered at a rate of from about 0.1 μg per hour to 200 μg per hour for a period of at least a week

5. The method of claim 3, wherein said dosage form comprises a depot.

6. The method of claim 5, wherein said depot comprises a non-polymeric high viscosity material.

7. The method of claim 6, wherein said high viscosity material comprises sucrose acetate isobutyrate.

8. The method of claim 3, wherein said dosage form comprises a biodegradable implant.

9. The method of claim 8 wherein said biodegradable implant comprises a polymer, and wherein said polymer is selected from the group consisting of: poly(DL lactide-co-glycolide), polycaprolactone, polyglycolide, and combinations thereof.

10. The method of claim 3, wherein said drug delivery device comprises a microsphere formulation, and wherein said microsphere formulation comprises a polymer selected from the group of poly(DL-lactide-co-glycolide), polycaprolactone, polyglycolide, and combinations thereof.

11. The method of claim 3, wherein said dosage form comprises a pump.

12. A method for increasing bone density in a subject, the method comprising: implanting in said subject a sustained release dosage form, said sustained release dosage form comprising a non-polymeric depot, and a bisphosphonate, and administering said bisphosphonate from said non-polymeric depot into said subject, for a period of at least a week, in an dose sufficient to cause a measurable increase in bone density.

13. An implantable dosage form comprising a drug delivery device and a bisphosphonate, wherein said drug delivery device is selected from the group consisting of: a bioerodable implant, a depot, and a microsphere formulation.

14. The implantable dosage form of claim 13 wherein the drug delivery device comprises a non-polymeric high viscosity material.
